(19) United States
(12) Patent Application Publication
(10) Pub. No.: US 2023/0149098 A1
(43) Pub. Date: May 18, 2023

(54) TARGET APPARATUS FOR USE IN A SURGICAL NAVIGATION SYSTEM, SURGICAL NAVIGATION SYSTEM, AND METHOD FOR PRODUCING SUCH A TARGET APPARATUS

(71) Applicant: Carl Zeiss Meditec AG, Jena (DE)

(72) Inventor: Christian VOIGT, Abtsgmünd (DE)

(21) Appl. No.: 17/979,315

(22) Filed: Nov. 2, 2022

(30) Foreign Application Priority Data
Nov. 16, 2021 (DE) 10 2021 212 877.7

(51) Int. Cl. A61B 34/20; A61B 90/00

(52) U.S. Cl. CPC A61B 34/20; A61B 90/39; A61B 2034/2055

(57) ABSTRACT

The invention relates to a target apparatus for use in a surgical navigation system, comprising at least one marker, for pose determination both by way of a device for marker reference point-based pose determination using at least two image capturing devices for imaging the marker and by way of a device for marker shape-based pose determination using at least one further image capturing device for imaging the marker , the marker having a detection surface, the detection surface having at least one first region and at least one further region, wherein the first region is designed to be more reflective than the further region, at least one portion of the first region and at least one portion of the at least one further region forming a pattern for identifying the marker, and to a surgical navigation system and a method for producing the target apparatus.

The invention relates to a target apparatus for use in a surgical navigation system, to a surgical navigation system, and to a method for producing such a target apparatus.

A surgical navigation system allows pose information to be provided in relation to surgical and other instruments in the operation region. By way of example, the location of a surgical instrument in the operation region of an operating theater, in particular the location relative to a patient, can be communicated to a surgeon by means of a surgical navigation system. At least one image capturing device that images what are known as markers can be used within the scope of an optical pose determination. The markers can be attached to the instrument whose pose is intended to be determined. Stereo tracking systems, amongst others, are known; these use two image capturing devices, which are generally arranged with different orientations of their optical axes and at a predetermined distance from one another, for producing image representations. A center of the image region in the image representations, in which a marker is imaged by an image capturing device, is determined for the purposes of pose determination. On the basis of the image coordinates of this center in the image representations produced by the two image capturing devices, it is possible to use triangulation methods to determine a spatial position of the marker in a reference coordinate system. For imaging purposes, such stereo tracking systems often use infrared radiation and markers that reflect this infrared radiation. For example, a marker can be designed as a sphere and the image capturing device can capture the radiation reflected by the sphere.

Additionally, methods for pose determination that evaluate the shape of an imaged marker in order to determine information about the latter’s pose in a reference coordinate system are known. By way of example, information about a distance of the marker from the image capturing device and optionally also about an orientation relative to an optical axis of the image capturing device can be determined from the dimensions of the image region in which a marker is imaged. Such methods allow the determination of such pose information from the evaluation of a single image representation produced by an image capturing device and are used, inter alia, for what is known as monocular tracking. As a rule, markers that are suitable for the application of these methods do not have a retroreflective design. US 2017 / 258531A1 has disclosed, for example, markers with a checkerboard-like light/dark pattern, with properties of the pattern being used to determine the pose. By way of example, WO 2008/056180 A2 has disclosed a sphere with a pattern, comprising a number of rings around the sphere. On the basis of an alignment of the sphere in relation to an image capturing device, a pose of the sphere can be determined from the pattern thus formed.

Whether a sphere or any other type of marker is suitable for use in a surgical system depends, in particular, on the properties of the utilized image capturing device and/or on the expectable distance between marker and image capturing device. Expressed differently, the type of image capturing device usable in an operating theater may be limited by the type or nature of a marker. This results in a number of conflicting aims when capturing a marker by means of an image capturing device, which are described in more detail below. Thus, one such conflicting aim consists in the choice of the exposure time of an image sensor of an image capturing device. On the one hand, the exposure time should be chosen such that an image representation of a marker is disturbed as little as possible by what are known as movement artifacts, with such artifacts arising in the case of long exposure times and on account of a movement of the marker during the exposure time. On the other hand, the exposure time should be long enough to capture sufficient luminous energy with the image sensor to thus render the marker capturable by the image sensor and imageable with a desired image quality. A further conflicting aim consists with regards to the choice of a size of the aperture. On the one hand, more light strikes the image sensor in the case of a larger aperture, making the capture of light reflected by the marker easier. On the other hand, there is a greater depth of field when capturing an object in space in the case of a smaller aperture. But a greater depth of field makes it easier to distinguish the marker from the surroundings, thus simplifying the pose determination. A further conflicting aim consists in the choice of the resolution of an image sensor. On the one hand, the achievable measurement accuracy within the scope of pose determination increases with increasing resolution. On the other hand, the size of the image sensor or the luminous energy in the captured region must be increased in the case of a higher resolution since the luminous energy capturable by a pixel per unit time might otherwise be too low to achieve an acceptable signal-to-noise ratio. In this context, an increase in the image sensor’s size is accompanied by increased costs in particular, especially since higher-quality optics are required if the size of the image sensor remains unchanged. As a rule, a light source is arranged in the operating theater to illuminate the marker. In particular, a light source may also be arranged on an image capturing device. As a rule, an arrangement of the light source on the image capturing device is required if retroreflective markers are used since the light emitted by the light source is only reliably reflected to the image capturing device in that case. The use of such a light source also gives rise to a conflicting aim. Although, on the one hand, the image representation of a marker is improved with an increased power of the light source since the latter then emits greater amounts of luminous energy, attempts are also made on the other hand not to increase the power of such a light source since an increased power of the light source is also accompanied by increased risk of possible damage to the human eye by light beam exposure, especially if the light source emits light in the spectrum not visible to the eye, for example as in the case of an infrared light source. Further, an increase in power of the light source is accompanied by increased power losses. Increased power losses yield greater heat losses, which must then be compensated by way of a cooling apparatus, for example, in order to ensure unchanging temperature conditions in the operating theater or in the image capturing device.

The prior art has disclosed markers for pose determination by means of tracking systems which use an image capturing device for capturing the marker. Additionally, markers suitable for detection by means of a tracking system with and without an image capturing device are known. Thus, US 2007 / 280508 A1 has disclosed what is known as a retro-grate reflector, which enables a position measurement using a single camera and a single marker. US 2017 1 238998 A1 describes a marker for use in a surgical navigation system, wherein the marker may have different sections for capture by a camera and by a 3-D scanner. US 2016 / 106515 A1 describes a system for use in osteotomy surgery, wherein reflective markers are used to determine the pose of at least three points on a human leg. US 2020 / 273199 A1 describes a method for pose determination between a thermal detection device and a 3-D camera, wherein at least one marker with at least two surface properties is used for pose determination, one surface scattering light diffusely and a second surface reflecting light specularly. CA 2907554 A1 has disclosed a marker which can be captured by a stereo camera or a non-stereo camera. However, one and the same embodiment of the marker is not identifiable by both camera systems. US 2020/0197098 A1 describes a marker with an uppermost layer that may have a visual print. This print may be interchangeable. Different regions of the print being able to have different optical properties, for example different reflectivities, is also disclosed. EP 3 212 104 B1 discloses a hybrid tracker, which comprises a target for optical tracking and sensor coils for electromagnetic tracking.

A technical problem arising is that of developing a target apparatus for use in a surgical navigation system, which allow a reliable and accurate pose determination by different optical devices for pose determination, with the aforementioned conflicting aims also being mitigated in particular.

The solution to the technical problem is provided by the subject matter having the features of the independent claims. Further advantageous configurations of the invention are evident from the dependent claims.

A basic concept of the invention lies in designing a marker in such a way that a simple and reliable determination of a reference point of the imaged marker is rendered possible for pose determination by a first device for pose determination, with the detection surface simultaneously allowing an accurate determination of the pose and a reliable identification of the marker by a further device for pose determination.

Therefore, a target apparatus for use in a surgical navigation system is proposed, comprising at least one marker, for pose determination both by way of a device for marker reference point-based pose determination, which comprises at least two image capturing devices for imaging the marker, and by way of a device for marker shape-based pose determination, which comprises at least one further image capturing device for imaging the marker. The marker has a detection surface, the detection surface having at least one first region and at least one further region. The detection surface is two-dimensional. The detection surface can be a flat surface or a curved surface. The detection surface is designed such that it is at least partially optically capturable by the first and/or further image capturing device(s), that is to say the detection surface is imageable when the marker is arranged in a captured region in particular. Each region of the detection surface is assigned at least one partial area of the detection surface. The detection surface and/or the regions or the partial areas of the regions are preferably contiguous.

A position and/or an orientation of the marker is determined in a reference coordinate system for the purposes of pose determination of the target apparatus. In this case, the reference coordinate system preferably comprises three perpendicular axes. The reference coordinate system preferably is a stationary, global coordinate system, although it may also be designed differently therefrom. A marker or a part of the marker may be assigned a marker coordinate system, in particular a Cartesian coordinate system. The latter may comprise two axes which, for example, span a plane of the detection surface and are oriented orthogonally with respect to one another, with a third axis being oriented normally to the detection surface. An origin of the coordinate system can be arranged at a reference point of the marker, for example a centroid of the area of the detection surface or of a region of the detection surface.

A pose, that is to say a position and/or orientation, of the marker relative to an instrument may be predetermined, for example by attaching the marker to a carrier that is connected to the instrument. Thus, the pose of the target apparatus or the marker can be determined for the purposes of pose determination of the surgical instrument, with, on account of the predetermined relative pose, a pose of the instrument then being able to be determined from the pose determined thus. The target apparatus may additionally comprise a carrier, the at least one marker being attached to the carrier. The target apparatus preferably comprises more than one marker, in particular at least three markers, which, in particular, are arranged, e.g., attached to the carrier, with a pose relative to one another that is known in advance. The carrier is preferably connected to a surgical instrument via an interface. The target apparatus may also be referred to as a target. If the target apparatus comprises a plurality of markers, it is also possible to determine a relative arrangement of the plurality of markers with respect to one another, in particular in the case of a marker reference point-based pose determination of each marker. This relative arrangement is able to encode additional information, for example an identity of the target apparatus.

Thus, the pose of the marker can be determined by means of various devices for pose determination. A first device is a device for marker reference point-based pose determination and comprises at least two image capturing devices, which enable the aforementioned stereoscopic pose determination. An image capturing device of the first device for pose determination is also referred to as first image capturing device below. In this case, the first device can be used to determine the position of the target apparatus should the latter comprise one marker or a plurality of markers. The first device can be used to determine the orientation of the target apparatus should the latter comprise at least three markers. A further device is a device for marker shape-based pose determination and comprises at least one, more particularly exactly one, image capturing device. The image capturing device(s) of the further device for pose determination is/are also referred to as further image capturing device(s) below. A first and/or further image capturing device may comprise at least one image sensor, for example a CCD or CMOS sensor. An image sensor produces an image representation of a captured region on the basis of light beams which strike the image sensor from the captured region. An image representation of a captured region generated thus is two-dimensional. An image capturing device may be assigned an internal coordinate system. Within the scope of pose determination, it is possible to determine a unique spatial relationship between the coordinate systems of the marker and the image capturing device, in particular in the internal coordinate system. This can then be transformed into the reference coordinate system by way of a transformation which, for example, is determined by way of a calibration. The image capturing devices of the first and the further device for pose determination differ from one another. By way of example, the image capturing devices of the various devices for pose determination may differ in terms of the resolution of a respective image sensor, in terms of the range of the light spectrum capturable by the image capturing device, and/or in terms of the size of an image sensor or an aperture. The number of image capturing devices used in the various devices for pose determination may also differ.

A marker reference point-based pose determination denotes a pose determination in which - as explained above - a reference point of the imaged marker, that is to say of the image region in which the marker is imaged, or coordinates of the reference point is/are determined. In particular, the reference point can be a center or centroid, more particularly a centroid of the area, of the imaged marker, which is depicted as a spot of light in the image representation. As already mentioned at the outset, information about a pose of the marker in a reference coordinate system can then be determined on the basis of the reference point by way of methods known to a person skilled in the art.

A marker shape-based pose determination denotes a pose determination in which a shape or a shape property, for example a dimensional property, of the imaged marker, that is to say of the image region in which the marker is imaged, is determined and evaluated taking account of a shape/shape properties known in advance. By way of example, a circle can be determined as a shape and a radius or diameter can be determined as a shape property. If a marker forms a pattern or comprises the latter, it is also possible to determine a shape or a property of the pattern for the purposes of pose determination within the scope of a marker shape-based pose determination. A relative pose of the regions that form the pattern, that is to say, for example, a relative pose of the first and the further region or of portions of these regions, with respect to one another may also be a property of a pattern. By way of example, a pattern of a marker for shape-based pose determination may be in the form of, e.g., an annular pattern, a QR code, a reticle pattern or a checkerboard pattern. Further, a marker for shape-based pose determination may also be in the form of what is known as an ArUco marker, with the embodiment of such markers and the pose determination being described, inter alia, in the document “ArUco: An efficient library for detection of planar markers and camera pose estimation,”, retrievable from https://docs.google.com/document/d/1QU9KoBtjSM2kF6ITOjQ76xqL7HOTEtXriJX5kwi9K gc/edit#heading=h.tmqpaefeqg1d (Effective: Nov. 15, 2021).

In this case, an actual shape of a marker and its property (properties) are known in advance, for example in the form of a reference shape or reference property. By way of example, a marker or a part thereof may be in a circular form, with the marker then being assigned the circle as a reference shape and/or a reference radius. Additionally, a marker or a part thereof may be embodied as an annular pattern, with the marker or a part thereof being assigned the annular pattern as a reference pattern and/or a predetermined reference inner radius and reference outer radius. The property (properties) known in advance allow information about a pose of the marker in a reference coordinate system to then be determined from the imaged shape or its properties by way of methods known to a person skilled in the art. By way of example, if a shape is detected in a captured region of an image capturing device, the device for pose determination can determine at least one property of the captured shape and compare this to a reference variable. Then, the spatial pose of the marker can be determined from the property and the reference variable, in particular by using known imaging properties of the image capturing device.

The pose determination comprises an evaluation of the image representation of the captured marker, with in particular an image region in which the marker is imaged being identified. Further, the evaluation within the scope of marker reference point-based pose determination comprises the determination of the coordinates of the reference point and the evaluation within the scope of marker shape-based pose determination comprises the determination of the shape or at least one property of the shape. By way of example, the evaluation can be implemented by executing an algorithm, with the first or further device for pose determination being able to comprise a computing device for execution purposes. In this case, each of the first and the further device may comprise a respective computing device, with the computing devices differing from one another. Additionally, the first and the further device may comprise a common computing device. The computing device may comprise a microcontroller or an integrated circuit, or be embodied as such.

According to the invention, the first region is designed to be more reflective than the further region. This may mean that the first region is designed to be more reflective for at least one predetermined wavelength or for light with wavelengths from at least one predetermined wavelength range, in particular for light from the infrared wavelength range, that is to say comprising wavelengths from 780 nm (inclusive) to 1 mm (inclusive). Additionally, the first region can be designed to be more reflective than the further region for all wavelengths. Further, at least a portion of the first region or the entire first region and at least a portion of the at least one further region or the entire further region form a pattern for identifying the marker. Thus, an identity, more particularly a biunique identity, of the marker can be assigned to a pattern. This identity, in turn, may be assigned the aforementioned shape, known in advance, or property, known in advance, of the marker. Additionally, the identity can be assigned further properties, for example an optical property, an assignment to a predetermined medical instrument to which the marker has been fastened, a date of manufacture or information that differs therefrom. A corresponding assignment may be stored, for example, in the form of a database in a memory device. Thus, if a pattern, in particular with at least one predetermined property, is detected or identified in the image representation, the identity assigned to the pattern can be determined on the basis of the assignment, for example be retrieved from the database. Further, on the basis of the assignment, the shape, known in advance, or its property and/or a further property can be retrieved in defined fashion, for example from the database, in particular in order to enable the marker shape-based pose determination. Expressed differently, an identity of the marker can be determined for marker shape-based pose determination, with at least one property of the shape, known in advance, then being determined on the basis of the identity, with the spatial pose then being determined on the basis of the at least one property known in advance, in particular by way of a comparison with a corresponding property (determined in image-based fashion) of the imaged marker. The pattern for identification purposes or a part thereof may form a pattern for pose determination or a part thereof.

Thus, the first region of the detection surface may have a configuration that is suitable for marker reference point-based pose determination. The first region may also comprise a plurality of portions, with the totality of the portions embodying the shape for marker reference point-based pose determination or the part thereof. The further region may also comprise a plurality of portions. Thus, the totality of the first region and further region may have a configuration that is suitable for marker shape-based pose determination. The portions of the first and the further region may be contiguous or disjoint portions. By way of example, the first region may be embodied in the form of an annulus or may comprise a portion embodied thus, with this shape being imaged by the first image capturing device(s) as a light spot whose center is easily and reliably determinable. In that case, the further region of the detection surface may be embodied as a circle, for example, which is arranged within the annulus. Thus, the arrangement of the first and the further region yields a pattern made of the annulus of the first region and the circle, with this pattern being identifiable in the image representation from the further image capturing device(s) and - as explained above - allowing a determination of the identity of the marker. In this case, the first and the further region preferably cover the detection surface in its entirety. In particular, the first region is distinguishable from the further region, or the portions are distinguishable from one another, in the image representation, at least in the image representation from the further image capturing device(s). A distinguishability of the first region from the further region can be provided by a corresponding brightness or color contrast, which is produced by a color scheme of the first and the further region. It is also conceivable that a transition region is located between the first and the further region, said transition region for example being colored in such a way that there is a smooth brightness or color change from the first to the further region.

Expressed differently, the first region enables marker reference point-based pose determination and the totality of the first region and the further region enables the identification of the marker and optionally the marker shape-based pose determination of the marker. Hence, the proposed marker can be used for both types of pose determination, in particular within the scope of a simultaneous pose determination. Thus, it is conceivable that a marker is captured from a plurality of (viewing) angles or by means of a plurality of image capturing devices, for example in order to reduce or resolve errors and/or ambiguities within the scope of pose determination. By way of example, a marker reference point-based pose determination can advantageously be verified by means of marker shape-based pose determination, or vice versa, without another marker having to be used to this end. In this case, a relative pose of the image capturing devices with respect to one another may be determined in advance. Naturally, it is also possible to determine a change in the pose of the marker over time, for example as translations along axes (forward/backward, up/down, and left/right), and/or in combination with changes in the orientation by way of rotations about the axes of the reference coordinate system. The proposed target apparatus is further advantageous in that it can be used in a plurality of operating theaters with differing devices for pose determination, without the marker or the image capturing device having to be adapted to new requirements in this case. Then again, it is also conceivable that the target apparatus is used in an operating theater which has available both a device for marker reference point-based pose determination and a device for marker shape-based pose determination. Thus, the target apparatus according to the invention can advantageously increase the user-friendliness of a surgical navigation system and/or increase an accuracy when determining the pose of a surgical instrument.

Naturally, the marker may also be suitable for capture by further tracking systems, in particular for capture by a computed tomography, magnetic resonance imaging and/or ultrasound-based tracking system. By way of example, the first and/or the further region of the detection surface or another part of the marker could form or comprise a reference structure that is capturable by means of a computed tomography and/or ultrasound-based tracking system. Additionally, the first and/or the further region or another part of the marker could contain a contrast agent in order to be capturable by means of a magnetic resonance imaging tracking system.

In a further embodiment, the first region is designed to be retroreflective and the further region is designed to scatter diffusely or be absorbent, in particular for the aforementioned wavelength(s). Should light strike the first region, it is cast back in directed fashion, that is to say it is retroreflected. If the navigation system, in particular the device for marker reference point-based pose determination, comprises a light source for example, in particular an infrared light source, the emitted light beams can be retroreflected by the first region of the marker. Consequently, they are cast back to the first image capturing device directly counter to the capturing direction. The light source can be arranged and/or designed in such a way that it emits light beams coaxially with an optical axis of the first image capturing device, for example as a ring light source. As a result, less luminous energy is sufficient for the purposes of capturing the marker since the light beams emitted by the light source are cast directly back to the first image capturing device and are not, or only partially, scattered diffusely in space. By way of example, a first region embodied thus enables the use of a first image capturing device with a small aperture and/or with short exposure times, since less light is required for capturing a retroreflective region. Further, it is possible to use a light source with less power. The conflicting aims described at the outset are advantageously mitigated as a consequence. Advantageously, simple locating of the reference point in the image representation is also rendered possible since the light spot caused by the marker is easily detectable in the image representation on account of the high intensity. Should light strike the further region, it is cast back in non-directed fashion, that is to say it is diffusely scattered. Thus, the pattern formed by the first and the further region can be captured more reliably by means of the further image capturing device since the distinguishability of the two regions has been improved. Consequently, what advantageously also arises on account of the different reflection properties of the first and the further region is an improved distinguishability of the two regions in the image representation, which improves a reliability of the identification in particular.

In a further embodiment, the first and/or further region has/have a symmetric design. Preferably, both regions and/or an overall region comprising the first and the further region have a symmetric design, further preferably both regions are assigned the same axis (axes) of symmetry and/or point of symmetry. A symmetric design of the first and/or the further region may mean that the partial area of the detection surface assigned to the first or the further region is assigned at least one axis of symmetry and/or point of symmetry, in particular if the region or the partial area of the region is captured from a plan view, for example from a direction oriented normally with respect to the detection surface. The point of symmetry may be a centroid of the area of the first and/or the further region, an axis of symmetry may run through this centroid of the area. The symmetric design advantageously increases the reliability and accuracy of marker reference point-based pose determination. The first and/or the further region particularly preferably has a point symmetric design as this enables a particularly reliable and accurate marker reference point-based pose determination. Additionally, marker shape-based pose determination may be simplified by the symmetry since there may be a small number of reference variables, which as described above are assigned to a shape. By way of example, the shape of a rectangle has two axes of symmetry, with two reference variables being required to describe the rectangle, preferably the height and the width of the rectangle. In comparison therewith, in particular all side lengths and interior angles are required as reference variables to describe a quadrilateral without an axis of symmetry. Advantageously, this can reduce computational outlay when determining the pose of the marker.

However, it is naturally also conceivable that there is an embodiment in which the first and/or the further region does not have a symmetric design. That is to say that no axis of symmetry can be assigned to at least one region. In particular, such asymmetry can be used to resolve ambiguities in the marker pose determination since for example the asymmetry of a region allows the marker to be assigned a unique reference orientation, for example relative to the first and/or the further image capturing device.

In a further embodiment, the further region is completely or partially surrounded by the first region, or vice versa. It is possible that the further region or a portion of the further region is partially or completely surrounded by the first region or a portion of the first region. Alternatively or cumulatively, the first region or a portion of the first region is partially or completely surrounded by the further region or a portion of the further region.

This can improve the image-based detectability of the regions. Preferably, a correspondingly distinguishable coloration of the two regions is chosen in order to attain an advantageous contrast by way of the coloration, with the coloration also comprising the provision of the regions with different brightnesses. This makes it easier to determine a pattern formed by the first and the further region. Overall, the described embodiment facilitates a detection of the marker pattern in an advantageous manner such that, in particular, a reliability within the scope of identification is improved.

In a further embodiment, the device for marker reference point-based pose determination is designed as, or comprises, a stereo camera system and/or the device for marker shape-based pose determination is designed as, or comprises, a monoscopic tracking system. Consequently the marker, in particular the first region, may be designed for capture by one of the at least two image capturing devices of the stereo camera system. Such an image capturing device, which may also be referred to as a stereo tracking camera, forms one image capturing device of a stereo camera pair for imaging objects from different positions and/or different angles. Consequently, the marker is advantageously designed for three-dimensional pose determination by a stereo camera system, which enables a pose determination with great accuracy. Alternatively or cumulatively, the first and the further region is designed for capture by the (exactly one) image capturing device of the monoscopic camera system. Only a single image representation may be required for pose determination of a marker when a monoscopic tracking camera is used. The device for pose determination then for example uses a segmentation algorithm which detects the first and the further region in a single captured monoscopic image representation, with then at least the shape of the first region being used for pose determination and the pattern formed by the first and the further region being used for identification purposes. Thus, an advantage that arises is that the marker is reliably detectable both by a monoscopic tracking camera and by a stereo tracking camera. In particular, the marker may be designed such that, within a predetermined working distance range, the first region is able to be imaged by at least five pixels using a stereo tracking camera with imaging properties known in advance. This advantageously renders it possible, for example, to be able to implement a reference point determination with a sufficient accuracy within the entire captured region of the stereo tracking camera. This ensures a pose determination of the marker within the entire captured region of the stereo tracking camera. Further, the marker may be designed such that, within a predetermined working distance range, the totality of the first and the further region is able to be imaged by at least ten pixels using a monoscopic tracking camera with imaging properties known in advance. This advantageously renders it possible to resolve edges, for example, with a sufficient accuracy in order to be able to capture in particular a transition from the first to the further region with sufficient accuracy. This ensures a reliable pattern determination and identification of the marker over the entire captured region of the monoscopic tracking camera.

In a further embodiment, the first and the further region are arranged centered with respect to one another. An arrangement of the first and the further region centered with respect to one another may mean that a partial area of the detection surface assigned to the first region and a partial area of the detection surface assigned to the further region have a common center of the area. This center of the area may be a point of symmetry. Additionally, an origin of the marker coordinate system, for example, may be arranged in the common centroid of the area. In particular, this advantageously improves a pose determination of the marker since the common center of the area can be used for detection of the marker in the case of both marker reference point-based and marker shape-based pose determination.

In a further embodiment, the first and/or further region has/have a circular, ellipsoid or ring-shaped design. A circular, ellipsoid or ring-shaped design of the first and/or the further region is advantageous in that a partial area of the detection surface assigned to the first or the further region has a point of symmetry and at least two axes of symmetry within the scope of the definition of symmetry provided above. For example, the shape of a circle or ring has an infinite number of axes of symmetry, with one reference variable being sufficient to describe a circular shape or an annular shape, for example a radius. A circular region is distinguished from a ring-shaped region in the sense that no other region is arranged within a circular region. If a region has a ring-shaped design, this denotes, in particular, an annular design, with for example an inner and an outer radius being required as reference variables to describe an annular shape. Further, the shape of an ellipse has two axes of symmetry, and so two reference variables are required to describe an ellipse-shaped region, for example a length of the major axis and a length of the minor axis. The described design of the first and/or the further region advantageously enables a reduction in the computational outlay when determining the pose of the marker, in particular for detecting a shape and/or a pattern.

In a further embodiment, the further region has smaller dimensions and/or the further region covers a smaller partial area of the detection surface than the first region. By way of example, the further region may have a further radius, which is smaller than a radius of the first region. This yields distinguishable reference variables or region-specific properties, facilitating the detection of a marker. The smaller dimensions or partial area of the further region vis-à-vis the first region yields the advantage that a distinguishability between the two regions is increased, in turn enabling a more reliable detection of a reference point assigned to the first region and/or of a pattern assigned to the first and the further region.

In a further embodiment, the further region is, or portions of the further region are, not resolvable by the first image capturing device, in particular if the marker is arranged spaced apart from the first image capturing device at at least one predetermined working distance. Not resolvable denotes the fact that the first image capturing device cannot image the further region in an image representation of the marker or cannot image the latter with a desired number of pixels, for example with a number less than one, two, three, or four pixels, and this further region consequently is not detectable in the image representation of the first image capturing device. Expressed differently, the pattern formed by the first and the further region cannot be detected/identified in the image representation of the first image capturing device. However, it is possible that the further region or a portion of the further region is resolvable by the further image capturing device if the marker is arranged spaced apart from the first image capturing device at the predetermined working distance. In particular, this can be achieved by virtue of the fact that the first image capturing device has a lower resolution than the further image capturing device, for example a resolution lower by a factor of at least two, at least five, or at least ten. It is also conceivable that a size of one aperture or, especially in the case of a stereo tracking camera, a size of a plurality of apertures of the first image capturing devices is chosen such that the further region is not resolvable by the first image capturing device, for example because insufficient light from the further region reaches the image sensor of the first image capturing devices on account of the chosen size of the aperture. An advantage arising is that the first region need not be distinguished from the further region for marker reference point-based pose determination because the further region is not resolvable by the first image capturing device. This enables a simplified and/or fast marker reference point-based pose determination.

In a further embodiment, the target apparatus comprises at least two markers, with each detection surface of the markers spanning a respective plane, with the at least two planes being arranged so as not to be parallel to one another or so as to be parallel and spaced apart from one another. The markers may differ from one another, that is to say the markers for example have different sizes and/or the markers differ from one another in terms of the design of the first and/or the further region. Each of the at least two markers can be assigned a marker coordinate system, in particular a Cartesian coordinate system. By way of example, the markers can be arranged at/on a carrier, with a first marker being able to be attached to a first part of the carrier and a further marker being able to be attached to a further part of the carrier. In this case, the first and the further part of the carrier may be oriented in different planes, with the different planes intersecting. Thus, the detection surfaces of the first and the second marker span at least two non-parallel planes, i.e., two intersecting planes. Naturally, other arrangements of the at least two markers which allow non-parallel planes to be spanned are also conceivable. The arrangement in non-parallel planes and, in particular, in parallel but spaced apart planes advantageously enables the resolution of ambiguities and reduces the errors when determining the pose of the target apparatus, especially since there is a redundancy in the detection of the markers and consequently in the determination of the target apparatus pose. The arrangement in non-parallel planes advantageously gives rise to a capturability of the markers from different capturing/illumination directions, and hence a directional redundancy. An arrangement in parallel but spaced apart planes advantageously enables unique pose determination from a capturing direction with as few markers as possible.

In a further embodiment, the first and/or the further region of the detection surface encode information, in particular information that differs from the identity of the marker. By way of example, information can be encoded by the first and/or the further region if the first and/or the further region has a feature, determinable in image-based fashion, which is assigned the information, with the feature for example arising from an asymmetric design of the first and/or the further region. Thus, the shape assigned to the first region and/or the pattern assigned to the first and the further region may be designed such that the feature is inherent to the shape and/or the pattern. In particular, a feature determined in image-based fashion may be compared to a reference feature in order to determine the information. By way of example, the information can be information about a property of the region/marker, for example about a size, an optical property, an assignment to a predetermined medical instrument to which the marker has been fastened, a date of manufacture or information that differs therefrom. Advantageously, the information can consequently also be determined without the marker being identified. By way of example, the information may be encoded by an ArUco pattern or a QR code pattern. An advantage arising is that information in addition to the pose information can easily be determined when a marker is detected.

In a further embodiment, the target apparatus comprises at least three markers, particularly exactly three markers. A redundancy and an accuracy when determining the pose is advantageously increased and/or the risk of an ambiguous pose determination is reduced if the target apparatus comprises at least three markers. In particular, it is possible to determine a pose of each marker of the target apparatus and, subsequently from this, a resultant pose of the target apparatus, especially if the relative pose of the at least three markers is known in advance. It is possible to use, for example, a triangulation method for pose determination, in particular by means of the device for pose determination. The at least three markers can be arranged with a defined spatial relationship to one another by way of a carrier, which is also referred to as a navigation star. The carrier can be part of the target apparatus. The target apparatus or the carrier can be arranged on a surgical instrument for the purposes of determining the latter’s pose.

In a preferred embodiment, the at least three markers are arranged with respect to one another, in particular spaced apart from one another, such that no ambiguity arises during the pose determination in space. There is ambiguity, in particular, if different poses can be assigned to a target apparatus or a marker. A relative arrangement of the markers with respect to one another may be predetermined, with each marker being individually capturable by the first and the further image capturing device. In this case, a spacing can be chosen such that two of the at least three markers have a minimum distance of 15 mm from one another. Additionally, a further spacing can be chosen such that two of the at least three markers have a minimum distance of 25 mm from one another. This can uniquely determine an orientation of the target apparatus. In this case, a spacing can be determined as a distance of the centers of the areas of the respective detection surfaces from one another. An advantage arising is that there are no ambiguities when determining the pose of the target apparatus, improving the reliability and accuracy of pose determination.

Further, a surgical navigation system comprising a target apparatus according to one of the embodiments described in this disclosure is proposed, wherein the navigation system comprises at least one first and at least one further image capturing device. A surgical navigation system designed thus is advantageous in that the disadvantage known from the prior art does not occur and the conflicting aims within the scope of determining the pose of the target apparatus, described at the outset, are mitigated. The navigation system may comprise a surgical microscope, with the device for marker shape-based pose determination or at least a part thereof, in particular the further image capturing device, being able to be integrated in the surgical microscope, for example in a microscope body with optical elements for microscopic imaging.

Further proposed is a method for producing a target apparatus according to any one of the preceding embodiments, wherein a less reflective medium, in particular a diffusely scattering or absorbing medium, is applied to a more reflective detection surface, in particular a retroreflective detection surface, or wherein a more reflective medium is applied to a less reflective detection surface, in particular a diffusely scattering or absorbing detection surface. Consequently, a target apparatus formed using a production method described in this disclosure is also described. Thus, a more reflective surface/medium is a surface/medium which is designed to be stronger than the less reflective surface/medium. In particular, the production can be implemented by virtue of providing a blank. This blank may have a detection surface which, for example, is retroreflective or diffusely scattering at least in part. The blank, in particular the detection surface thereof, may be produced from a film or a laminate. The regions of the detection surface with a retroreflective design may for example be coated with a film or a laminate having retroreflective beads or pigments. Then, continuing, a diffusely scattering medium is applied to an at least partially retroreflective detection surface. Alternatively, a reflective medium is applied to an at least partially diffusely scattering detection surface. The applied medium can be an ink, a (further) film, or a (further) laminate. However, other media are also suitable for application. In particular, the medium can be produced in a printing method, for example by printing a blank film. The application of the medium can be enabled, in particular, by way of the medium adhering to the detection surface. In particular, the medium is applied in such a way that the detection surface with the first and/or the further region is formed. The target apparatus can be produced in the operating theater, especially just before or during the implementation of a surgical intervention. Thus, the target apparatus can advantageously be produced directly when required, in particular before or during an operation and/or according to individual requirements.

Alternatively, a more reflective ink, in particular a retroreflective ink, is applied to a first region of the detection surface and a less reflective ink, in particular a diffusely scattering or absorbing ink, is applied to a further region of the detection surface. The respective inks are printed onto the blank of the target apparatus such that the first and/or the further region form as a result of printing. An advantage arising is that the above-described blank need not already have an at least partially more reflective or less reflective property.

Further alternatively, a detection surface (or a part thereof) is more reflective, in particular retroreflective, with a region of the detection surface being irradiated in thermally sensitive and/or photosensitive fashion using a radiation source, with the more reflective property of this region changing and this region being less reflective, more particularly being diffusely scattering or absorbent, following the irradiation. To this end, the detection surface may be coated with a photoresist in particular, the latter reacting in a thermally sensitive and/or photosensitive fashion. Some of the photoresist can be cured or removed by irradiation. This may yield a change, for example in the retroreflective property of an irradiated region. By way of example, a laser unit which radiates pulses of light on the region of the detection surface to be irradiated in a targeted fashion may be a suitable radiation source. The irradiated region may then form the first and/or the further region at least in part. An advantage arising is that no additional materials are required to produce the target apparatus. In particular, the blank can be packaged in sterile fashion in light-transmissive packaging. Irradiation and production of the target apparatus can be implemented directly on the sterile blank through the packaging, especially directly in the operating theater.

The invention is explained in more detail on the basis of exemplary embodiments. In the figures:

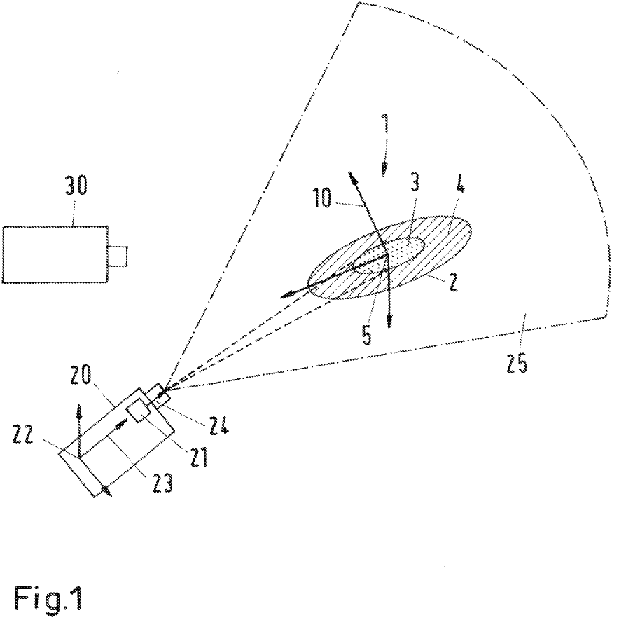

FIG. 1: shows a target apparatus according to the invention in the captured region of a first image capturing device,

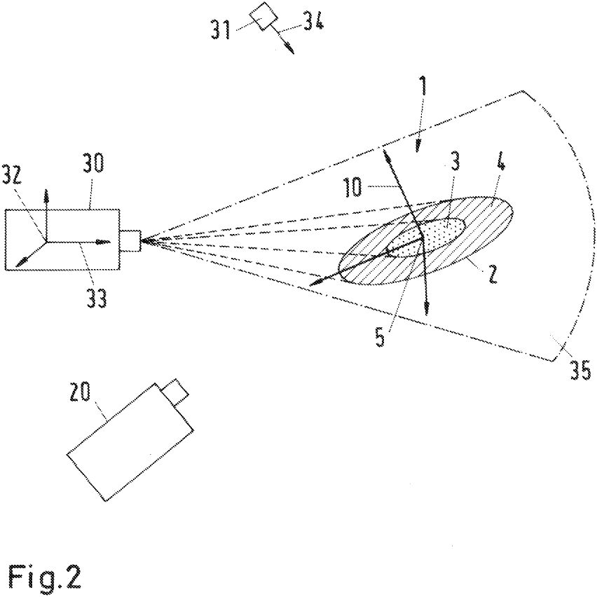

FIG. 2: shows a target apparatus according to the invention in the captured region of a further image capturing device,

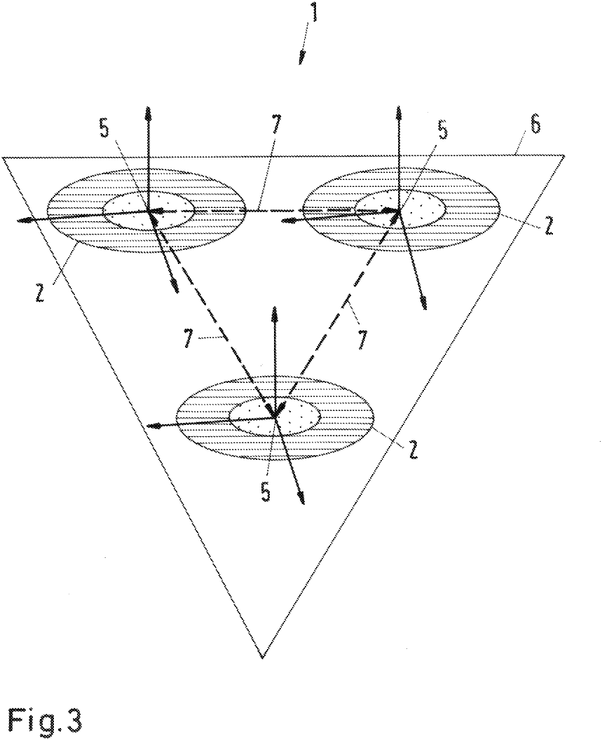

FIG. 3: shows a target apparatus according to the invention comprising three markers,

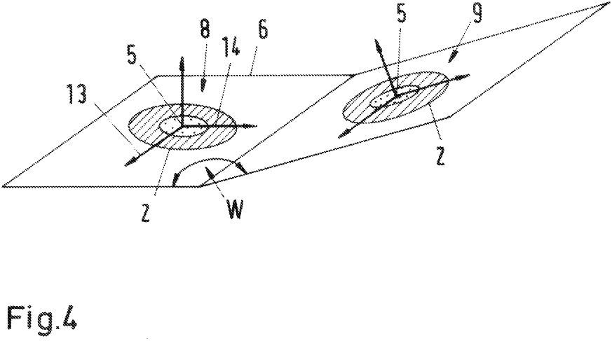

FIG. 4: shows a target apparatus according to the invention comprising two markers,

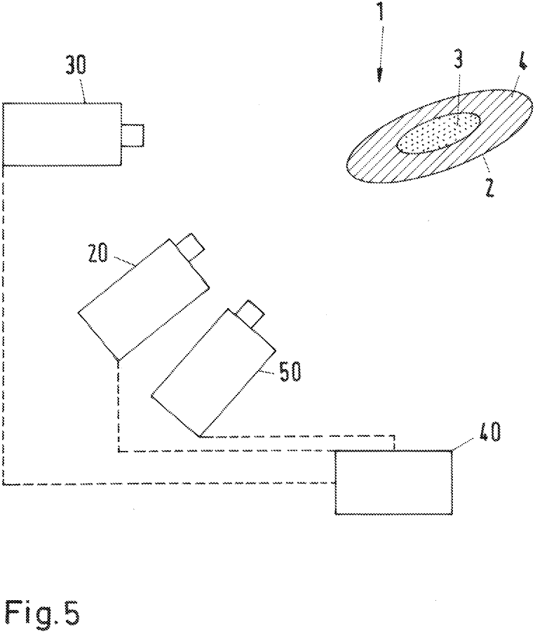

FIG. 5: shows a surgical navigation system according to the invention,

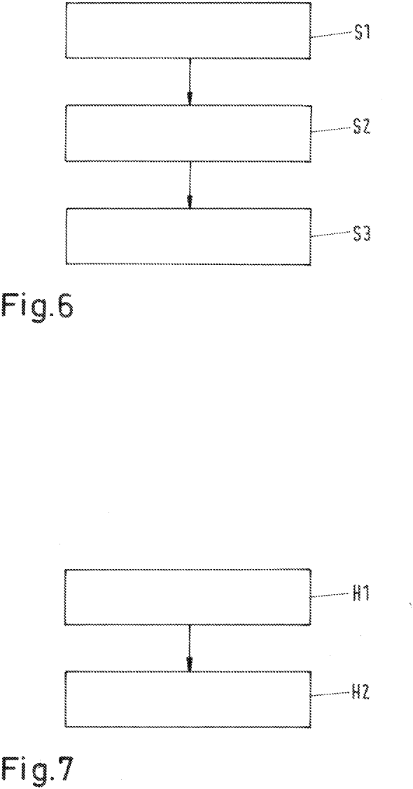

FIG. 6: shows a schematic flowchart for a method for pose determination,

FIG. 7: shows a schematic flowchart for a production method according to the invention, and

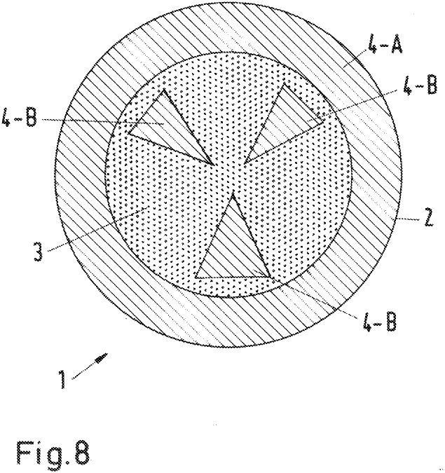

FIG. 8: shows a target apparatus comprising a marker.

Identical reference signs hereinafter denote elements having identical or similar technical features. FIGS. 1 and 2 show a target apparatus 1 according to the invention comprising a marker 2. In the embodiment of the target apparatus 1 shown, the latter has a detection surface which is divided into a first region 4 and a further region 3. The further region 3 has a circular design and is labeled by a dotted surface, with this surface being a diffusely scattering or light-absorbing surface. The first region 4 is labeled by a hatched surface which is a retroreflective surface and designed in annular fashion. The first and the further region 4, 3 form a pattern, with the first region 4 completely surrounding the further region 3. In FIG. 1, the target apparatus 1 is arranged in a captured region 25 of a first image capturing device 20. The captured region 25 is depicted by a dash-dotted line. A light source 21 of the first image capturing device 20, which emits light coaxially with a capturing direction 24 of the image capturing device 20, is also depicted. This light source may be an infrared light source. The light is radiated into the entire captured region 25. If a light beam emitted by the light source 21 strikes the first region 4, it is cast directly back to the image capturing device 20 on account of the retroreflective coloration. Thus, a small amount of luminous energy is already sufficient for imaging the first region 4 of the marker 2 since the light beams emitted by the light source 21 are not diffusely scattered but retroreflected by the first region 4. In particular, the image capturing device 20 can be configured such that light beams diffusely scattered in the captured region 25 are captured to a much lesser extent than the retroreflected light beams. This facilitates the detection of the first region 4 by the first image capturing device 20 since only retroreflected light beams are captured. The first image capturing device 20 has an internal coordinate system 22, which may serve as a reference coordinate system for pose determination. In this case, a longitudinal axis 23 may be oriented parallel to the capturing direction 24 of the coordinate system 22, with the capturing direction corresponding to a direction of the optical axis of the first image capturing device 20. Further, the first image capturing device 20 is part of a device for marker reference point-based pose determination (not depicted here). This device for pose determination uses the coordinate system 22 in order to determine a pose, that is to say a position and/or orientation, of the target apparatus 1 relative to the reference coordinate system. Also imaged here is a further image capturing device 30 which is different from the first image capturing device 20 and which is also suitable for pose determination; see FIG. 2. The first region 4 is assigned an annulus as a shape. A center of the image region in which the annulus is imaged by the first image capturing device 20 may form a reference point for marker reference point-based pose determination, with the pose of the marker being determined on the basis of the image coordinates of the reference point. Thus, in particular, the pose of the reference point can be determined as pose of the marker. It is also possible that the first image capturing device 20 is one of two image capturing devices for stereoscopic imaging of the target apparatus 1 and consequently the device for marker reference point-based pose determination comprises a further first image capturing device, not depicted here, in addition to the first image capturing device 20. In this case, the center of the first region 4 imaged by the two image capturing devices of the device for marker reference point-based pose determination can be determined in each corresponding image representation, with a spatial pose of the target apparatus 1 being determined on the basis of the pose of the centers in the image representations by way of known triangulation methods.

In FIG. 2, the target apparatus 1 is arranged in a captured region 35 of the further image capturing device 30, with the captured region 35 likewise being delimited by a dash-dotted line. The captured region 35 and the captured region 25 may differ from one another. The further image capturing device 30 has an internal coordinate system 32 with a longitudinal axis 33, with the longitudinal axis 33 specifying a capturing direction of the further image capturing device 30. The internal coordinate system 32 may likewise serve as a reference coordinate system. Shown further is a further light source 31, which emits light non-directionally into the captured region 35. In this case, the emission direction 34 of the further light source 31 is non-parallel, in particular, to the longitudinal axis 33. The further image capturing device 30 is different from the first image capturing device 20 and part of a device for marker shape-based pose determination. The image capturing device 30 produces an image representation of the first and the further region 3, 4, which is analyzed by means of the device for shape-based pose determination. Capturing the first and the further region 3, 4 by the further image capturing device 30 is schematically depicted by four dashed lines. By way of known methods of image processing and image analysis, the device for shape-based pose determination, in particular the evaluation device 40 thereof, is able to identify, for example, a pattern comprising an ellipse or a circle and an elliptical ring or annulus in the image representation generated, with then an identity assigned to this pattern and hence marker 2, in particular in biunique fashion, being determined. Further, the lengths of the major and minor axes of the ellipse and the elliptical ring or the radii of the inner circle and annulus can be determined in image-based fashion. Depending on the identity, it is then possible to determine, more particularly retrieve, reference properties of the marker 2 known in advance, for example a radius of the outer circle and/or of the inner circle known in advance. The properties with which the first region 4 and/or the further region 3 are imaged in various possible poses, that is to say positions and/or orientations, by means of the further image capturing device 30 may also be known in advance, for example be determined by way of a calibration, and/or be retrievable, in particular the radii or lengths of the major and minor axes with which the inner circle, imaged in circular or elliptical form, and/or the outer circle, imaged in circular or elliptical form, of the annulus are imaged. As a result, the current pose of the marker 2 can be determined by comparing the properties determined in image-based fashion with the pose-specific properties known in advance, which were determined on the basis of the identity.

FIG. 3 shows a target apparatus 1 according to the invention comprising three markers 2. In terms of their configuration, the markers 2 correspond to the markers described in FIGS. 1 and 2. The markers 2 are arranged on a carrier 6, for example by adhesive bonding on a surface of the carrier 6. The carrier 6 is part of the target apparatus 1 and may be attached to a surgical instrument such that a pose of the surgical instrument is determinable by determining a pose of the target apparatus 1. The markers 2 are arranged at a predetermined spacing 7 from one another. The spacing can be chosen so that the markers 2 do not conceal one another in the captured region of an image capturing device and an accuracy during pose determination is increased. In this case, the spacing 7 is defined as a distance between the centers of the areas of the respective detection surfaces. In the centroids of the areas, a marker coordinate system 5 is arranged per marker 2.

This can ensure that it is possible to determine the pose of each marker 2 arranged on the carrier 6. Further, ambiguities when determining the pose of the target apparatus 1 can be resolved by determining the poses of the markers 2.

FIG. 4 shows a target apparatus 1 according to the invention comprising two markers 2. The markers 2 are arranged on a carrier 6, with the carrier 6 being subdivided into a first carrier part 8 and a second carrier part 9. The two carrier parts 8, 9 of the carrier 6 are arranged at an angle W with respect to one another, with the angle W differing from 0° or 180°. One marker 2 is attached to a surface of the first part 8 and another marker 2 is attached to a surface of the second part 9. If the markers 2 are detected by means of a device for pose determination, a marker coordinate system 5 can be assigned to each marker 2, as described above. By assigning the marker coordinate system 5, a plane lying in the detection surface of the marker 2 is spanned by the longitudinal and transverse axes 13, 14. On account of the non-parallel arrangement of the parts 8, 9 of the carrier 6, the planes spanned thus intersect at the angle W, the planes are consequently non-parallel. It is mentioned that the perspective views of the target apparatus 1 chosen in FIGS. 1 to 4 should be understood to be schematic examples of image representations of the target apparatus 1 capturable by the first and/or the further image capturing device 20, 30 and therefore may naturally also be configured differently.

FIG. 5 shows a surgical navigation system according to the invention. A target apparatus 1 comprising a marker 2 is located in a captured region 25, 35 (see FIGS. 1 and 2) of the first and/or the further image capturing device 20, 30, the marker 2 having a detection surface with a first and a further region 4, 3. Shown further is a further first image capturing device 50, with the two first image capturing devices 20, 50 forming the two image capturing devices of a stereo camera system which - as explained above -enables a stereoscopic, marker reference point-based determination of the pose of the target apparatus 1 or of the marker 2 by evaluating the image representations produced by the two first image capturing devices 20, 50. To this end, the target apparatus 1 is likewise located in the captured region (not depicted here) of the further first image capturing device 50. The image capturing devices 20, 30, 50 are data-connected to an evaluation device 40. The evaluation device 40 can be part of the first and/or the further device for pose determination. If an image representation of the marker 2 is generated by the first image capturing devices 20, 50 and/or further image capturing device 30, such an image representation can be evaluated by the evaluation device 40 for the purposes of determining a pose of the target apparatus 1.

FIG. 6 shows a flowchart of a method for pose determination by means of a target apparatus 1 according to the invention, wherein, in a step S1, an image representation of the target apparatus 1 or at least of a first region 4 of a marker 2 is generated using the first image capturing devices 20, 50 of a stereo camera system (see FIG. 5) and/or an image representation of the first and a further region 4, 3 is generated using a further image capturing device 30. In a step S2, the image representations of at least the first region 4, which are generated by the first image capturing devices 20, 50, are evaluated by means of an evaluation device 40 and a marker reference point-based determination of the pose of the marker 2 is carried out in a reference coordinate system. In a step S3, an identity of the marker 2 is determined by evaluating the pattern formed by the first and the further region 4, 3 and a marker shape-based determination of the pose of the marker 2 in the reference coordinate system is subsequently carried out on the basis of the identity. Consequently, marker reference point-based pose determination is implemented independently of marker shape-based pose determination; in particular, these two types of pose determination are based on image representations produced by different image capturing devices 20, 30, 50. Thus, the two types of pose determination form alternatives for pose determination. In particular, the pose determined in marker reference point-based fashion can be verified by means of the pose determined in marker shape-based fashion, or vice versa, without this requiring the use of another marker. It is also conceivable that a resultant pose is determined from the pose determined in marker reference point-based fashion and the pose determined in marker shape-based fashion, for example using a method of data fusion.

In this context, the depicted sequence of the second and the third step S2, S3 is not mandatory; position determinations implemented in these steps S2, S3 may also be carried out simultaneously or in reversed order.

FIG. 7 shows a flowchart of a production method according to the invention for producing a target apparatus 1. A blank of a marker 2 or a target apparatus 1 is provided in a first step H1. This blank may have a detection surface which is retroreflective or diffusely scattering at least in part. The blank, in particular the detection surface thereof, may be produced from a film or a laminate. In one form of a production step H2, a diffusely scattering medium is applied to a retroreflective detection surface of the blank. Alternatively, a reflective medium is applied to a diffusely scattering detection surface of the blank. It is also conceivable that, within the scope of production step H2, a retroreflective ink is applied to a first region 3 of the detection surface of the blank and a diffusely scattering ink is applied to a further region 4 of the detection surface. The respective inks are printed onto the blank of the target apparatus 1 such that the first and/or the further region 4, 3 form as a result of printing. It is further possible that a further region 3 of the detection surface is irradiated in thermally sensitive and/or photosensitive fashion using a radiation source, with a retroreflective property of the further region 3 changing and the further region 3 diffusely scattering after the irradiation. To this end, the detection surface of the blank may be coated with a photoresist in particular, the latter reacting in a thermally sensitive and/or photosensitive fashion. An advantage arising is that no additional materials are required to produce the target apparatus 1. The irradiation and production of the target apparatus can be implemented directly on the sterile blank, directly in the operating theater in particular, for example by means of a laser.

FIG. 8 shows a target apparatus 1 with a marker 2, which has a first region 4 which is depicted by hatching and which is composed of portions 4-A, 4-B. A first portion 4-A has an annular design and encompasses a further region 3, with three further portions 4-B of the first region 4 being arranged in the further region 3, that is to say the further region 3 encompasses the three further portions 4-B of the first region 4. In this case, what is depicted is that the further portions 4-B of the first region 4 have a triangular design. Alternatively, these can be formed like a circular sector.

### List of Reference Signs

| | |
|---|---|
| 1 | Target apparatus |
| 2 | Marker |
| 3 | Further region |
| 4 | First region |
| 5 | Marker coordinate system |
| 6 | Carrier |
| 7 | Spacing between markers |
| 8 | First part of a carrier |
| 9 | Second part of a carrier |
| 10 | Vertical axis of a marker coordinate system |
| 13 | Longitudinal axis of a marker coordinate system |
| 14 | Transverse axis of a marker coordinate system |
| 20 | First image capturing device |
| 21 | Light source for the first image capturing device |
| 22 | Coordinate system of the first image capturing device |
| 23 | Longitudinal axis of the coordinate system of the first image capturing device |
| 24 | Capturing direction of the first image capturing device |
| 25 | Captured region of the first image capturing device |
| 30 | Further image capturing device |
| 31 | Light source for the further image capturing device |
| 32 | Coordinate system of the further image capturing device |
| 33 | Longitudinal axis of the coordinate system of the further image capturing device |
| 34 | Emission direction |
| 35 | Captured region of the further image capturing device |
| 40 | Evaluation device |
| H1 | First production step |
| H2 | Second production step |
| S1 | First method step |
| S2 | Second method step |
| S3 | Third method step |
| W | Angle |

1. A target apparatus for use in a surgical navigation system, comprising at least one marker, for pose determination both by way of a device for marker reference point-based pose determination using at least two image capturing devices for imaging the marker and by way of a device for marker shape-based pose determination using at least one further image capturing device for imaging the marker, the marker having a detection surface, the detection surface having at least one first region and at least one further region,
wherein
the first region is designed to be more reflective than the further region, at least one portion of the first region and at least one portion of the at least one further region forming a pattern for identifying the marker.

2. The target apparatus as claimed in claim 1, wherein the first region is designed to be retroreflective and the further region is designed to scatter diffusely or absorb radiation.

3. The target apparatus as claimed in claim 1, wherein the first and/or further region have/has a symmetric, more particularly point symmetric design.

4. The target apparatus as claimed in claim 1, wherein the further region is completely or partially surrounded by the first region, or vice versa.

5. The target apparatus as claimed in claim 1, wherein the device for marker reference point-based pose determination is designed as, or comprises, a stereo camera system and/or the device for marker shape-based pose determination is designed as, or comprises, a monoscopic tracking system.

6. The target apparatus as claimed in claim 1, wherein the first and the further region are arranged centered with respect to one another.

7. The target apparatus as claimed in claim 1, wherein the first and/or further region have/has a circular, ellipsoid or ring-shaped design.

8. The target apparatus as claimed in claim 1, wherein the first region has smaller dimensions and/or covers a smaller partial area of the detection surface than the further region.

9. The target apparatus as claimed in claim 1, wherein the further region or portions of the further region is/are not resolvable by the first image capturing device, in particular if the marker is arranged spaced apart from the first image capturing device at a predetermined working distance.

10. The target apparatus as claimed in claim 1, wherein the target apparatus comprises at least two markers, with each detection surface of the markers spanning a respective plane, with the at least two planes being arranged so as not to be parallel to one another or so as to be parallel and spaced apart from one another.

11. The target apparatus as claimed in claim 1, wherein the first and/or further region of the detection surface encode information.

12. The target apparatus as claimed in claim 1, wherein the target apparatus comprises at least three markers.

13. The target apparatus as claimed in claim 12, wherein the at least three markers are arranged with respect to one another, in particular spaced apart from one another, such that no ambiguity arises during the pose determination in space.

14. A surgical navigation system comprising a target apparatus as claimed in claim 1, the navigation system comprising at least two first and at least one further image capturing device.

15. A method for producing a target apparatus as claimed in claim 1 , wherein a less reflective medium is applied to a more reflective detection surface or wherein a more reflective medium is applied to a less reflective detection surface or wherein a more reflective ink is applied to a first region of the detection surface and a less reflective ink is applied to a further region of the detection surface or wherein a detection surface is reflective, with a region of the detection surface being irradiated in thermally sensitive and/or photosensitive fashion using a radiation source, with the reflective property of this region changing and this region being less reflective following the irradiation.
